(19) United States
(12) Patent Application Publication
(10) Pub. No.: US 2008/0268076 A1
(43) Pub. Date: Oct. 30, 2008

(54) Composition for Inhibiting Acyl-Coa:Cholesterol Acyltransferase

(76) Inventors: Young kook Kim, Daejeon (KR); Mun chual Rho, Daejeon (KR); Hyun sun Lee, Daejeon (KR); Seung woong Lee, Daejeon (KR); Oh eok Kwon, Daejeon (KR); Mi yeon Chung, Daejeon (KR); Jung ho Choi, Daejeon (KR); Mi sook Dong, Seoul (KR)

Correspondence Address: Casimir Jones, S.C., 440 Science Drive, Suite 203, Madison, WI, 53711, US

(21) Appl. No.: 11/994,936

(22) PCT Filed: Jul. 8, 2005
(86) PCT No.: PCT/KR06/02673

§ 371 Date: Mar. 13, 2008

(30) Foreign Application Priority Data
Jul. 8, 2005 (KR) 10-2005-0061830

(51) Int. Cl. A61K 36/67; A61K 31/36; A61K 31/4025; A61K 31/16; A61P 9/00

(52) U.S. Cl. 424/734; 514/466; 514/422; 514/627

(57) ABSTRACT

Disclosed herein is a composition having activity of inhibiting acyl-CoA: cholesterol acyltransf erase, which comprises retrofractamide A, pipercide, piperrolein B, piperchabamide D, pellitorin or combinations thereof, or a pharmaceutically acceptable salt thereof . The composition is effective for preventing and treating vascular diseases, such as hyperlipidemia, arteriosclerosis, etc.

### TECHNICAL FIELD

The present invention relates to a composition having ability to inhibit acyl-CoA:cholesterol acyltransferase. More particularly, the present invention relates to a composition for inhibiting acyl-CoA:cholesterol acyltransferase, comprising as an active ingredient a Piper nigrum L. extract, a compound separated from the extract, or a pharmaceutically acceptable salt thereof.

### BACKGROUND ART

Vascular disease occurs mainly due to hyperlipidemia. It ranks highly among all diseases causing death. Therefore, medication for the treatment and prevention of vascular diseases is required.

According to Heider's study, the body is known to demand exogenous cholesterols, which are supplied thereto by the uptake of foods, as well as endogenous cholesterols, which are synthesized in the liver [Heider J. G. 1986. Agents which inhibit cholesterol esterification in the intestine and their potential value in the treatment of hypercholesterolaemia., J. R. Prous Science Publishers, 423-438]. However, excessive ingestion of triglycerides and cholesterol leads to hyperlipidemia, a condition which is characterized by excessively high levels of cholesterol or triglycerides in the blood, and which is known to be a major cause of arteriosclerosis. These diseases or conditions are due to the abnormal metabolism of lipoproteins during the formation, transport, and/or degradation of lipoproteins. Epidemiological investigation has shown that most ischemic heart diseases are mainly due to coronary atherosclerosis and that an increase in serum cholesterol level is a factor playing an important role in the incidence and progress thereof. Reports by Goldstein et al. and Komai teach methods of reducing levels of serum cholesterol by inhibiting the absorption of cholesterol in the small intestine and the biosynthesis of cholesterol in the liver and by promoting the secretion of bile acid [Goldstein J. L. and S. M. Brown 1990. Regulation of the mevalonate pathway:Nature 33 425-430, Komai T. and Y. Tsujita 1994. Hepatocyte selectivity of HMG-CoA reductase inhibitors: DN & P, 7: 279-288]. Medicaments currently available for decreasing serum cholesterol levels are exemplified by pravastatin and simvastatin, manufactured by Daiichi Sankyo, Japan, and Merck, U.S.A., respectively, which are both biologically modified from compactin inhibit cholesterol biosynthesis in the liver, and which both occupy the greatest percentages of the market and are being popularized at the highest rate. The medical mechanism of these medicaments is based on the inhibition of 3-hydroxy-3-methyl glutary Co-A reductase, which is involved in an intermediate step of cholesterol biosynthesis in the liver. Grunler's study revealed that the use of HMG Co-A reductase inhibitor for a long period of time has a negative effect on the production of ubiquinone, dolichol, haem A, farnesylated proteins, and cholesterol derivatives, such as steroid hormones, vitamin D, bile acid, lipoproteins, etc., which must be produced in the side pathway of cholesterol biosynthesis after the formation of mevalonate, and are essential for the body [Grunler J., J. Ericsson and G. Dalloner 1994. Branch-point reactions in the biosynthesis of cholesterol, dolichol, ubiquinone and prenylated proteins: Biochim. Biophys, Acta 1212, 259-277]. According to Willis' study, long-term use of HMG Co-A reductase inhibitor results in decreasing the biosynthesis of coenzyme Q, which plays an important role in heart function and immune function, thereby are having a dangerous effect on patients with arteriosclerosis or heart diseases [Willis R. A., K., Folkers. J. L. Tucker, C. Q. Ye, L. J. Xia, and H. Tamagawa. 1990. Lovastatin decreases coenzyme Q levels in rats: Proc. Natl. Acad. Sci. USA, 87, 8928-8930].

Currently available medical agents for hyperlipidemia are either inhibitors of cholesterol biosynthesis in the liver or anionic exchangers which are associated with bile acid and thus inhibit the re-absorption of cholesterol in the large intestine. Both of them are clinically used, but there is need for a novel medicament for hyperlipidemia, which can be used without limitation and has a reliable therapeutic mechanism with little or no side effects. Sliskovic reported that ACAT inhibitor is effective for the prevention and treatment of hyperlipidemia [Sliskovic D. R. and A. D. White 1991. Therapeutic potential of ACAT inhibitors as lipid lowering and antiatherosclerotic agents: Trends in Pharmacol. Sci. 12:194-199]. Particularly, intensive attention has been paid to the development of an ACAT inhibitor, which is evaluated as a hyperlipidemia therapeutic directly involved in a novel preventive mechanism against the occurrence of arteriosclerosis. ACAT is an enzyme known to be implicated in the acylation of cholesterol, thus participating in the absorption of cholesterol in the small intestine, the synthesis of VLDL (very low density lipoprotein) in the liver, and the accumulation of esterified cholesterol.

Many college institutes and commercial institutes, such as those of pharmacy companies, have researched hyperlipidemia therapeutics, and some of them have succeeded in developing effective therapeutics. In most cases, however, research and study have been focused on the development of ACAT inhibitors as the next-generation agents for preventing hyperlipidemia safely and reliably. Most of the ACAT inhibitors researched thus far are synthetic chemicals, based on urea, amide or phenol, like those developed in Warner Lambert, Pfizer, Yamanouchi, etc. [Matsuda K. 1994. ACAT inhibitors as antiatherosclerosis agent: compounds and mechanisms. 14, John Wiley & Son, Inc., 271-305]. In order to develop ACAT inhibitor precursors having novel structures, extensive research has been conducted using microbial materials. With the success of the Kitasato Institute of Japan in the structural identification of purpactin as a start [Tomoda H., H. Nishida, R. Masuma, J. Cao, S. Okuda and S. Omura 1991. Purpactins, new inhibitor of acyl-CoA: cholesterol acyltransferase produced by Penicillium purpurogenum I. Production, isolation and physico-chemical and biological properties: J. Antibiotics 44:136-143], various novel microbial ACAT inhibitors have been discovered, including epi-cohliquinone A of Daiichi Sankyo, Japan [Japanese Pat. Laid-Open Publication No. Hei 4-334383, 1992], acaterin of Tokyo University of Agriculture and Technology [Naganuma S., K Sakai, K. Hasumi and A. Endo 1992. Acaterin, a novel inhibitor of acyl-CoA: cholesterol acyltransferase produced by Pseudomonas sp. A92: J. Antibiotics 45:1216-1221], helminthosporol [Park J. K., K. Hasumi and A Endo 1993. Inhibitors of acyl-CoA:cholesterol acyltransferase by Helminthosporol and its related compounds: J. Antibiotics 46:1303-1305], lateritin [Hasumi K., C. Shinohara, T. Iwanaga and A. Endo 1993. Lateritin, A new inhibitors of acyl-CoA:cholesterol acyltransferase produced by Gibberella lateritium IFO 7188: J. Antibiotics 46:1782-1787], gypsetin [Shinohara C., K. Hasumi , Y. Takei and A. Endo 1994. Gypsetin, a new inhibitor of acyl-CoA: cholesterol acyltransferase produced by Nannizzia gypsea var. incurvata IFO 9228., I. Fermentation, isolation physico-chemical properties and biological activity: J. Antibiotics 47:163-167], enniatins of Kitasato Institute, Japan [Nishida H., X. H Huang, R. Masuma, Y. K. Kim and S. Omura 1992. New cyclodepsipeptides, enniatins D. E. and F produced by Fusarium sp. FO-1305: J. Antibiotics 45:1207-1214], glisoprenins [Tomoda, H. X. H, Huang, H. Nishida, R Masuma, Y. K. Kim and S. Omura 1992. Glisoprenins, new inhibitors of acyl-CoA: cholesterol acyltransferase produced by Gliocladium sp., I. Production. Isolation and physico-chemical and biological properties: J. Antibiotics, 45:1202-1206], pyripyropenes [Omura S., H. Tomoda, Y. K. Kim and H. Nishida 1993. Pyripyropenes, highly potent inhibitors of acyl-CoA: cholesterol acyltransferase produced by Aspergillus fumigatus: J. Antibiotics 46:1168-1169; Kim Y. K, H Tomoda, H. Nishida, T. Sunazuka, R. Obata, S. Omura 1994. Pyripyropenes, novel inhibitors of acyl-CoA: cholesterol acyltransferase produced by Aspergillus fumigatus., II. Structure elucidation of pyripyropenes A, B, C and D: J. Antibiotics 47:154-162], terpendols [Huang X. H, H. Tomoda, H. Nishida, R, Masuma and S. Omura 1995. Novel ACAT inhibitors produced by Albophoma yamanashiensis: J. Antibiotics 48:1-4], AS-183 of Kyowa Hakko, Japan [Kuroda K., M. Yoshida, Y. Uosaki, K. Ando, I. Kawamoto, E. Oishi, H. Onuma, K. Yamada and Y. Matsuda 1993. AS-183, a novel inhibitor of acyl-CoA: cholesterol acyltransferase produced by Scedosporium sp. SPC-15549: J. Antibiotics 46:1196-1202], AS-186 [Kuroda K., Y. Morishita, Y. Saito, Y. Ikuina, K. Ando, I. Kawamoto and Y. Matsuda 1994. AS-186, New inhibitor of acyl-CoA: cholesterol acyltransferase from Penicillium asperosporium KY1635: J. Antibiotics 47:16-22], GERI-BP-001 of the Korea Research Institute of Bioscience and Biotechnology [Jeong T. S., S. U. Kim, K. H Son , B. M Kwon, Y. K. Kim ,M. U. Choi and S. H. Bok 1995. GERI-BP001 compounds, New inhibitors of acyl-CoA: cholesterol acyltransferase produced by Aspergillus fumigatus F37: J. Antibiotics 48:751-756], GERI-BP-002 [Kim Y. K, H. W. Lee, K. H Son, B. M Kwon, T. S Jeong, D. H Lee, J H Shin, Y W. Seo , S. U. Kim, S. H. Bok 1996. GERI-BP002-A, Novel inhibitors of acyl-CoA:cholesterol acyltransferase produced by Aspergillus fumigatus F93: J. Antibiotics 49:31-36], and avasimibe of Pfizer, which are all attracting intensive attention [Heinonen T M., 2002. Acyl coenzyme A:cholesterol acyltransferase inhibition: potential atherosclerosis therapy or springboard for other discoveries?:Expert Opin Investig Drugs. 11:1519-1527].

Leading to the present invention, intensive and thorough research, conducted by the present inventors, into naturally occurring materials having activity of inhibiting ACAT resulted in the finding that amide-based compounds isolated from Piper nigrum L., including retrofractamide A, pipercide, piperrolein B, piperchabamide D, and pellitorin, have potent activity to inhibit ACAT and are effective for the prevention and treatment of hypercholesterolemia vascular diseases, such as hypercholesterolemia, hyperlipidemia, arteriosclerosis, etc.

### DISCLOSURE OF THE INVENTION

Therefore, it is an object of the present invention to provide a pharmaceutical composition having activity of inhibiting acyl-CoA: cholesterol acyltransferase, comprising an extract from Piper nigrum L., a compound selected from retrofractamide A, pipercide, piperrolein B, piperchabamide D, pellitorin and combinations thereof, or a pharmaceutically acceptable salt thereof.

It is another object of the present invention to provide a method for preparing an extract having activity of inhibiting acyl-CoA:cholesterol acyltransferase from Piper nigrum L.

It is a further object of the present invention to provide a method for separating an amide-based compound having activity of inhibiting acyl-CoA:cholesterol acyltransferase from the extract.

### BRIEF DESCRIPTION OF THE DRAWINGS

FIG. 1 shows spectrum data of the compound represented by Chemical Formula 1, including H-NMR (CDCl₃, 500.13 MHz), C-NMR (CDCl₃, 125.75 MHz), and FAB-Mass.

FIG. 2 shows spectrum data of the compound represented by Chemical Formula 2, including H-NMR (CDCl₃, 500.13 MHz), C-NMR (CDCl₃, 125.75 MHz), and FAB-Mass.

FIG. 3 shows spectrum data of the compound represented by Chemical Formula 3, including H-NMR (CDCl₃, 500.13 MHz), C-NMR (CDCl₃, 125.75 MHz), and FAB-Mass.

FIG. 4 shows spectrum data of the compound represented by Chemical Formula 4, including H-NMR (CDCl₃, 500.13 MHz), C-NMR (CDCl₃, 125.75 MHz), and FAB-Mass.

FIG. 5 shows spectrum data of the compound represented by Chemical Formula 5, including H-NMR (CDCl₃, 500.13 MHz), C-NMR (CDCl₃, 125.75 MHz), and FAB-Mass.

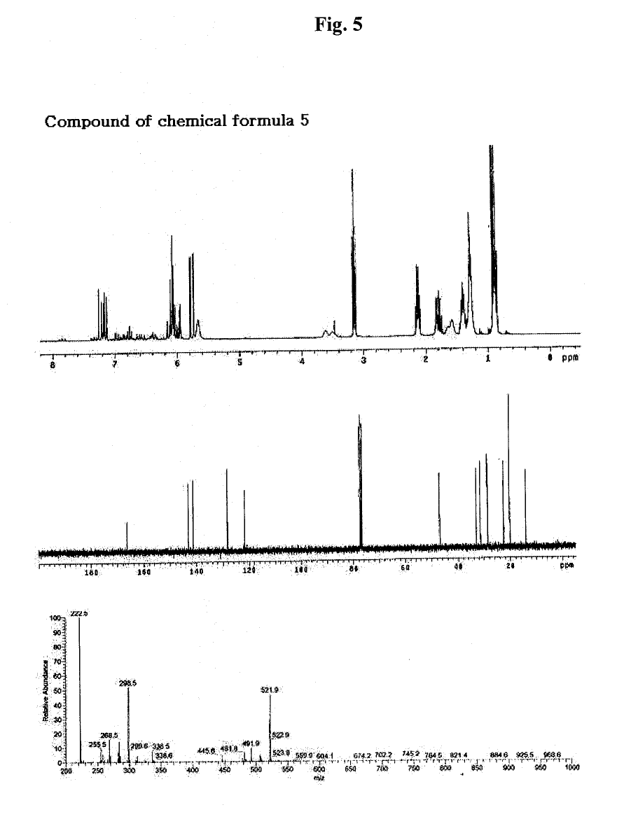

FIG. 6 shows % ACAT inhibition of the compounds of Chemical Formulas 1 to 5.

FIG. 7 shows the inhibitory activity of the compounds of Chemical Formulas 1 to 5 on ACAT in HepG-2 cells.

### BEST MODE FOR CARRYING OUT THE INVENTION

In accordance with an embodiment, the present invention pertains to a Piper nigrum L. extract having activity of inhibiting acyl-CoA:cholesterol acyltransferase, and a composition comprising the extract.

In accordance with another embodiment, the present invention pertains to a composition having activity of inhibiting acyl-CoA:cholesterol acyltransferase, comprising a compound selected from a group consisting of retrofractamide A, pipercide, piperrolein B, piperchabamide D, pellitorin, and combinations thereof, or a pharmaceutically acceptable salt thereof. retrofractamide A, pipercide, piperolein B, piperchabamide D, and pellitorine are preferably naturally occurring materials, extracted from Piper nigrum L. Alternatively, these compounds may be synthetic materials.

Acquired knowledge on the inhibitory activity of Piper nigrum L. extract on acyl-CoA:cholesterol acyltransferase (ACAT) led the present inventors to search for active ingredients in the extract. To accomplish this, a crude extract obtained by dissolving Piper nigrum L. in an organic solvent, such as alcohol, was fractioned in water and various organic solvents. All of the fractions were found to exhibit ACAT inhibition, with the highest inhibitory activity given to a chloroform fraction. This was subjected to various chromatographies to isolate active compounds. These were analyzed for structure and chemical properties through electron impact mass spectrometry, hydrogen nuclear magnetic resonance spectrometry, and carbon nuclear magnetic resonance spectrometry.

Results of the analyses revealed the active compounds retrofractamide A of Chemical Formula 1, pipercide of Chemical Formula 2, piperrolein B of Chemical Formula 3, piperchabamide D of Chemical Formula 4, and pellitorin of Chemical Formula 5. Nowhere has reference to the ACAT inhibition of these compounds been found in literature published prior to the present invention.

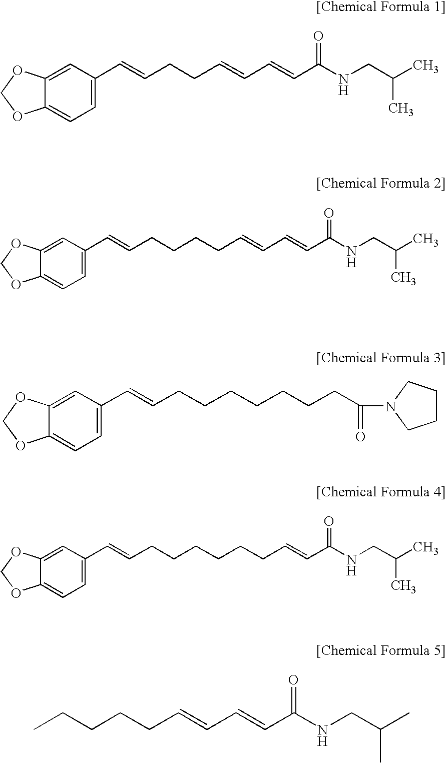

Acyl-coenzyme A:cholesterol acyltransferase (ACAT) is an integral membrane protein catalyzing the formation of cholesteryl esters from cholesterol and fatty acyl coenzyme A.

The term “acyl-CoA:cholesterol acyltransferase (ACAT) inhibition”, or corresponding phrases, as used herein, means that the enzymatic reaction leading to the formation of cholesteryl ester is blocked or becomes inefficient. The reaction that ACAT catalyzes is essential for the absorption of cholesterol in the intestine, the synthesis and secretion of apolipoprotein B (apoB), and the storage of cholesterol inside the cell. Thus, ACAT inhibition results in interruption of the absorption of cholesterol from foods in the intestine and VLDL in the liver, thus reducing the cholesterol level in serum.

Since the finding that ACAT has direct relation to serum levels of cholesterol, it has been studied as a therapeutic target for cholesterol-associated diseases. Based on the fact that selective ACAT inhibition results in a reduction in the serum level of cholesterol, effective treatment can be given to vascular diseases occurring in the brain, the heart, and peripheral vessels. For instance, the inhibition of the activity of ACAT is useful in preventing and treating hypercholesterolemia (Raal F J et al., Atherosclerosis. 2003 December; 171(2) :273-279), hyperlipidemia (kusunoki J., Arterioscler Thromb Vasc Biol. 2000 January; 20(1):171-178), atherosclerosis (Heinonen T M., Curr Atheroscler Rep. 2002 January; 4(1):65-70), arteriosclerosis (Heinonen T M., Expert Opin Investig Drugs. 2002 Nov. 11(11):1519-1527), coronary arteriosclerosis (Meynier A., Br J Nutr. 2002 May;87(5):447-458), and aortic aneurysms (Hiatt W R et al., Vasc Med. 2004 November; 9(4):271-277). In addition, ACTC is found to be involved in the production of Alzheimer's disease-associated amyloidal plaques, and thus this disease can be treated with ACAT inhibitors (Hutter-Paier B et al., Neuron. 2004 Oct. 14; 44(2): 227-238; Puglielli L et al., J Mol Neurosci, 2004; 24(1):93-96). Accordingly, selective inhibitors of ACAT can be used for the prevention and treatment of the aforementioned diseases as well as symptoms or complications thereof.

The term “prevention” of a disease as used herein indicates all actions for restricting or delaying the occurrence of the disease by administering the composition of the present invention. The term “treatment” of a disease as used herein indicates all actions for turning or changing conditions of the disease for the better or in a favorable direction through the administration of the composition.

The compounds used in the active ingredients of the composition according to the present invention can be isolated from organisms, and preferably from Piper nigrum L. Various organs, such as roots, stems, flowers, fruits, etc., of natural, hybrid or mutant plants as well as tissue cultures of the plants may be used to prepare the compounds. Also, they can be synthesized using a method known to those in the art.

As used herein, the term “pharmaceutically acceptable salt” means salts derived from pharmacologically or physiologically acceptable inorganic acids, organic acids and bases. Examples of suitable acids in the present invention include hydrochloric acid, hydrobromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, naphthalene-2-sulfonic acid, benzene sulfonic acid, etc. Salts derived from suitable bases are exemplified by salts of alkaline metals, such as sodium, salts of alkali earth metals, such as magnesium, and ammonium salts.

In another embodiment, the present invention provides a method for obtaining an extract from Piper nigrum L. and a method for separating certain compounds from the extract.

The extract from Piper nigrum L. can be obtained using water, organic solvents, or mixtures thereof. Preferably, after being dried for a predetermined period of time and crushed, Piper nigrum L may be subjected to an extraction method. Example of the useful extraction methods include, but are not limited thereto, cold precipitation, heat extraction, ultrasonic extraction, and cold extraction. As long as it destroys the active ingredients to a minimum extent, any extraction method may be used. The compounds having activity of inhibiting ACAT can be prepared by obtaining highly active fractions from the extract and separating them from the active fractions by, for example, chromatography.

Thus, the compounds can be prepared using a method comprising: extracting them from Piper nigrum L. into a medium such as water, organic solvents or mixtures thereof; fractioning the extract with a non-polar organic solvent; and purifying the content of the non-polar organic solvent by chromatography.

Examples of the organic solvent useful in the extraction of crushed plants include methanol, ethanol, isopropanol, butanol, ethylene, acetone, hexane, ether, chloroform, ethyl acetate, butyl acetate, dichloromethane, N, N-dimethylformamide (DMF), dimethylsulfoxide (DMSO), 1,3-butylene glycol, propylene glycol and mixtures thereof, with preference for alcohols and higher preference for lower alcohols such as methanol or ethanol.

In order to obtain fractions showing high activity from the primary extract of Piper nigrum L., water and organic solvents are employed. Of the organic solvents, non-polar organic solvents are preferred. Particularly preferable is n-hexane, ether, dichloromethane, chloroform, ethylacetate or mixtures thereof. In an example of the present invention, respective fractions were obtained in n-hexane, chloroform, ethylacetate and water. Of them, the chloroform fraction was found to have the highest activity (89%), with the lowest activity given to the water fraction (15%).

The non-polar solvent fraction, that is, the content dissolved in the non-polar solvent, is subjected one or more times to chromatography to isolate the active ingredients. Various chromatography columns and developing solvents may be suitably used.

In an example, compounds having the structures of Chemical Formulas 1 to 5 were isolated. Three weights of methanol were poured onto powdered Piper nigrum L. and allowed to stand for seven days at room temperature, followed by filtration and drying in a vacuum to concentrate them. The crude extract thus obtained was fractioned with n-hexane, chloroform, ethyl acetate or water. Respective fractions were concentrated in a vacuum and subjected to chromatography four times. Silica gel column chromatography (concentration gradient n-hexane:ethyl acetate=50/1˜0/100), reversed-phase column chromatography (ODS gel, methanol used), low pressure liquid chromatography (LPLC; LKB, methanol used), and high performance liquid chromatography (HPLC; YMC J'sphere ODS H-80 (250×20 mm)) were sequentially conducted to afford a total of five pure compounds.

Analysis through electron impact mass spectrometry, hydrogen nuclear magnetic resonance spectrometry, carbon nuclear magnetic spectrometry, etc. revealed the purified compounds to be amide compounds, identified as ractamideRetrof A of Chemical Formula 1, pipercide of Chemical Formula 2, piperolein B of Chemical Formula 3, piperchabamide D of Chemical Formula 4, and pellitorine of Chemical Formula 5. All the compounds corresponding to Chemical Formulas 1 to 5 are found to have activity of inhibiting ACAT with respective IC₅₀ values determined at 24.5, 3.7, 87.5, 11.5, 40.4 μM. Particularly, the inhibitory activity of pipercide of Chemical Formula 2 is 12 times as high as that of obovatol.

Having potent activity of inhibiting ACAT, the compounds of Chemical Formulas 1 to 5 are therapeutically useful for the prevention and treatment of cerebrovascular, cardiovascular, and peripheral vascular diseases on the basis of the grounds described above. In addition, the compounds show potent preventive and therapeutic activity for Alzheimer's disease. The composition of the present invention comprises non-synthetic, naturally occurring active compound(s), and thus is safe and can be administered for a long term with almost no toxicity or side effects. The composition is also effective for mammals which may suffer from cerebrovascular, cardiovascular or peripheral vascular diseases, such as cows, horses, sheep, pigs, goats, camels, antelopes, dogs, etc., as well as humans.

In accordance with another embodiment, therefore, the present invention provides a pharmaceutical composition effective for the prevention and treatment of vascular diseases, comprising an extract from Piper nigrum L., at least one of the compounds of Chemical Formulas 1 to 5, or at least one pharmaceutically acceptable salt thereof.

The pharmaceutical composition for the prevention and treatment of vascular disease in accordance with the present invention comprises the active ingredient selected from the compounds in an amount from 0.1 to 50 wt % in total based on the total weight of the composition. The composition may further comprise additives which are usually used to improve flavor, taste, appearance, and other non-medicinal properties. In addition, the composition may further comprise organic or inorganic additives selected from among vitamin B₁, B₂, B₆, C, and E, niacin, carnitin, betain, folic acid, pantothenic acid, biotin, zinc, iron, calcium, chrome, magnesium, and combinations thereof. The composition of the present invention may be used alone or in combination with a preexisting, therapeutically effective material.

The composition comprises a pharmaceutically acceptable carrier, and can be formulated into oral or non-oral dosage forms for humans and mammals.

For the formulation of the composition according to the present invention, diluents or expedients, such as fillers, thickeners, binders, wetting agents, disintegrants, and surfactants, are commonly used. Solid formulations for oral dosage include tablets, pills, powders, granules, and capsules. These solid formulations are prepared with the composition of the present invention in combination with at least one expedient such as starch, calcium carbonate, sucrose, lactose, or gelatin. In addition to the expedient, a lubricant, such as magnesium, stearate, talc, etc. can be used. Liquid formulations for oral administration include suspensions, internal solutions, emulsions, and syrups. In these liquid formulations, various expedients, such as wetting agents, sweeteners, and preservatives, as well as simple diluents such as water and liquid paraffin may be contained. Formulations for non-oral dosage may be typified by sterile aqueous solutions, non-aqueous solutions, suspensions, emulsions, lyophilized agents, and suppositories. For non-aqueous solutions and suspensions, vegetable oils such as propylene glycol, polyethylene glycol and olive oil, or injectable ester such as ethyloleate may be used.

The composition may be presented as unit-dose (single dose) or multi-dose forms, such as in sealed ampules and vials, or may be stored as a lyophilized form which requires only the addition thereto of a sterilized liquid vehicle, such as injection water, just before administration. In situ injections or suspensions may be prepared as sterile powders, granules or tablets.

In accordance with a further embodiment of the present invention, provided is a method for the prevention and treatment of vascular diseases, which comprises administering to a patient a composition containing as an active ingredient at least one selected from among the compounds of Chemical Formulas 1 to 5 or at least one salt thereof.

The term “a patient” as used herein means a mammal which suffers from a disease which can be alleviated when the ACAT inhibiting composition according to the present invention is administered thereto. In order to effectively prevent or treat vascular diseases, such as hypercholesterolemia, hyperlipidemia, atherosclerosis, arteriosclerosis, coronary arteriosclerosis and aortic aneurysms, the administration of the composition comprising an extract from Piper nigrum L. or at least one selected from among the compounds of Chemical Formulas 1 to 5 to patients in need thereof can be conducted. The composition may be administered in combination with a preexisting therapeutic agent therefor.

As used herein, the term “administration” means the introduction of a predetermined material into a patient using a suitable method. As long as it reaches a target tissue, any administration route, whether oral or non-oral, may be adopted. In addition, the composition of the present invention can be administered with the aid of an apparatus which allows the active ingredient to readily reach a target cell.

The composition of the present invention is administered in a pharmaceutically effective amount.

The term “pharmaceutically effective amount” as used herein means an amount sufficient to afford an optimal benefit/danger ratio during therapy therewith. This ratio is determined depending on various factors known in the medical field, including a patient's sex and age, the kind and severity of disease, drug activity, sensitivity, administration time, administration route, discharge ratio, administration time period, co-administered drugs, and others. The composition of the present invention may be administered alone or in combination with other therapeutics. The co-administration of the composition of the present invention with other therapeutics may be carried out simultaneously or sequentially. Single or multiple dosages are possible. It is important to use the composition in the minimum possible amount sufficient to obtain the greatest therapeutic effect without side effects. Preferably, the pharmaceutically effective amount of the composition of the present invention falls into the range from 1 to 10 mg/kg per dose for oral administration and the range from 1 to 5 mg/kg per dose for intravenous injection.

In still a further embodiment, the present invention provides a health food comprising an extract from Piper nigrum L. or a fraction isolated from the extract. As well as in the composition of the present invention, the extract from Piper nigrum L. or the fraction isolated therefrom may be used in a health food that can be convenient to take, thereby preventing vascular diseases or Alzheimer's disease at normal times.

The health food may be prepared using a method known to those in the art and may be in the form of tablets, granules, powders, beverages, etc.

A better understanding of the present invention may be obtained in light of the following examples which are set forth to illustrate, but are not to be construed to limit the present invention.

### EXAMPLE 1

### Isolation and Purification of Enzyme Inhibitor

After being washed with water and dried in a shady place, Piper nigrum L., purchased in a market located in Daejeon, Korea, was pulverized into powder using a pulverizer with blades. To 5 kg of the powdered Piper nigrum L. were added three weights of methanol, and the solution was allowed to stand for seven days at room temperature, followed by filtration. The filtrate was dried in a vacuum to yield a crude concentrated extract. This was dissolved in n-hexane, chloroform, ethylacetate and water to separate and purify active materials. Respective fractions of the crude extraction were assayed for ACAT inhibition activity. For this, part of each of the fractions was dried to give a test sample having a density of 1 mg/ml. The ACAT inhibition activity was measured to be 25% in the n-hexane fraction, 89% in the chloroform fraction, 55% in the ethylacetate fraction, and 15% in the water fraction. The total chloroform fraction with the highest ACAT inhibition activity was concentrated in a vacuum (157.7 g), and applied to silica gel column chromatography eluting with a step gradient eluent system consisting of n-hexane : ethylacetate=50/1˜0/100 to yield active fractions. A few fractions having the highest ACAT inhibition activity were pooled and purified through reversed-phase column chromatography on ODS gel using 50%, 60%, 70%, 80%, 90%, and 100% methanol as eluents. Fractions 4 and 5, which were higher in ACAT inhibition activity than others, were further purified through low pressure liquid chromatography (LKB) using 75% and 80% methanol at a flow rate of 6 ml/min and 8 ml/min respectively. Of the fractions thus obtained, No. 3 of Fraction 4 and Nos. 2 and 4 of Fraction 5, which were found to have higher ACAT inhibition activity than other Nos., were further subjected to high performance liquid chromatography (YMC J'sphere ODS H-80 (250×20 mm) eluting with 75% and 80% methanol at a flow rate of 4 ml/min and 6 ml/min to yield one active pure compound from No. 3 of Fraction 3 and four active pure compounds from No. 2 and 4 of Fraction 5. Detection of the ACAT inhibitors was performed using UV light at 254 nm and 210 nm 35 min after the start of the elution for No. 3 of Fraction 4, 31 min after the start of the elution for No. 2 of Fraction 5, and 43 min, 45 min and 53 min after the start of the elution for No. 4 of Fraction 5.

### EXAMPLE 2

### Structural Determination of Active Ingredients

Physicochemical properties of Compounds 1 to 5, extracted from Piper nigrum L. are as follows.

Compound 1

(1) Form: white powder

(2) Empirical formula and Molecular weight: C₂₀H₂₅NO₃, 327

(3) Electron impact mass spectrometry (70 eV): m/z(rel. int)=360 [M+Na]+

(4) H-NMR spectra[300 MHz, Chloroform-d₃, δ(ppm)]: 7.19 (1H, dd, J=15, 15.3 Hz, H-3), 6.87 (1H, br s, H-2′), 6.73 (1H, m, H-5′ and 6′), 6.30 (1H, d, J=15.3 Hz, H-9), 6.17 (1H, dd, J=9.9, 15.3 Hz, H-4), 6.10 (1H, m, H-5), 5.98 (1H, m, H-8), 5.93 (2H, s, H-7′), 5.77 (1H, d, J=14.7 Hz, H-2), 5.59 (NH, br s), 3.16 (2H, t, J=6.6 Hz, H-1″), 2.30 (4H, m, H-6 and 7), 1.79 (1H, m, H-2″), 0.93 and 0.91 (3H, s, H-3″ and 4 ″)

(5) C-NMR spectra [75 MHz, chloroform-d₃, δ(ppm)]: 20.10 (q, C-3″ and 4″), 28.59 (d, C-2″), 32.15 (t, C-7), 32.81 (t, C-6), 46.90 (t, C-2″), 100.92 (t, C-7′), 105.39 (d, C-2′), 108.20 (d, C-5′), 120.36 (d, C-6′), 122.23 (d, C-2), 127.66 (d, C-8), 128.77 (d, C-4), 130.15 (d, C-9), 132.04 (s, C-1′), 140.94 (d, C-3), 141.69 (d, C-5), 146.71 (s, C-3′), 147.91 (s, C-4′), 166.27 (s, C-1)

Compound 2

(1) Form: white powder

(2) Empirical formula and Molecular weight: C₂₂H₂₉NO₃, 355

(3) Electron impact mass spectrometry (70 eV): m/z(rel. int)=354 [M−H]+

(4) H-NMR spectra [300 MHz, chloroform-d₃, δ(ppm)]: 7.19 (1H, dd, J=14.4, 14.7 Hz, H-3), 6.88 (1H, br s, H-2′), 6.74 (1H, m, H-5¹), 6.73 (1H, br s, H-6′), 6.28 (1H, d, J=15.6 Hz, H-11), 6.13 (1H, dd, J=15.3, 15.3 Hz, H-4), 6.05 (1H, d, J=15 Hz, H-5), 6.02 (1H, d, J=15.9 Hz, H-10), 5.92 (2H, s, H-7′), 5.75 (1H, d, J=15.3 Hz, H-2), 5.56 (NH, br s), 3.16 (2H, t, J=6.6 Hz, H-1″), 2.17 (4H, m, H-6 and 9), 1.79 (1H, m, H-2″), 1.46 (4H, m, H-7 and 8),0.93 and 0.91 (3H, s, H-3″ and 4″)

(5) C-NMR spectra [75 MHz, chloroform-d₃, δ(ppm)]: 20.09 (q, C-3″ and 4″), 28.27 (t, C-7), 28.60 (d, C-2″),28.90 (t, C-8), 32.64 (t, C-9), 32.75 (t, C-6), 46.89 (t, C-1″), 100.88 (t, C-7′), 105.35 (d, C-2′), 108.18 (d, C-5′), 120.20 (d, C-6′), 121.89 (d, C-2), 128.38 (d, C-4), 128.92 (d, C-10), 129.52 (d, C-11), 132.32 (s, C-1′), 141.12 (d, C-3), 142.69 (d, C-5), 146.55 (s, C-4′), 147.90 (s, C-3′), 166.31 (s, C-1)

Compound 3

(1) Form: colorless oil

(2) Empirical formula and Molecular weight: C₂₁H₂₉NO₃, 343

(3) Electron impact mass spectrometry (70 eV): m/z(rel. int)=366 [M+Na]+

(4) H-NMR spectra [300 MHz, chloroform-d₃, δ(ppm)]: 6.88 (1H, br s, H-2′), 6.74 (1H, m, H-5′), 6.73 (1H, br s, H-6′), 6.27 (1H, d, J=15.3 Hz, H-9), 6.03 (1H, dt, J=15.9, 6.9 Hz, H-8), 5.92 (2H, s, H-7′), 5.75 (1H, d, J=15.3 Hz, H-2), 3.54 (2H, t, J=5.4 Hz, H-1″), 3.38 (2H, t, J=5.5 Hz, H-5″), 2.30 (2H, t, J=7.5 Hz, H-2), 2.16 (2H, q, J=6.6 Hz, H-7), 1.61 (4H, m, H-2″ and 4″), 1.54 (4H, m, H-4 and 3″), 1.45 (2H, m, H-6), 1.35 (4H, m, H-3 and 5)

(5) C-NMR spectra [75 MHz, chloroform-d₃, δ(ppm)]: 24.57 (t, C-3″), 25.37 (t, C-4″), 25.56 (t, C-4), 26.55 (t, C-2), 28.95 (t, C-5), 29.24 (t, C-6), 29.34 (t, C-3), 32.80 (t, C-7), 33.39 (t, C-2), 42.54 (t, C-1″), 46.67 (t, C-5″), 100.85 (t, C-7′), 105.34 (d, C-2′), 108.15 (d, C-5′), 120.14 (d, C-6′), 129.27 (d, C-8), 129.30 (d, C-9), 132.42 (s, C-1′), 5 146.48 (s, C-3′), 147.87 (s, C-4′), 171.37 (s, C-1)

Compound 4

(1) Form: white powder

(2) Empirical formula and Molecular weight: C₂₂H₃₁NO₃, 357

(3) Electron impact mass spectrometry (70 eV): m/z(rel. int)=380 [M+Na]+

(4) H-NMR spectra [300 MHz, chloroform-d₃, δ(ppm)]: 6.89 (1H, br s, H-2′), 6.83 (1H, dt, J=15.3, 7.5 Hz, H-3), 6.75 (1H, m, H-5′), 6.74 (1H, br s, H-6′), 6.28 (1H, d, J=15.9 Hz, H-11), 6.03 (1H, dt, J=15.3, 7.5 Hz, H-10), 5.93 (2H, s, H-7′), 5.75 (1H, d, J=15.3 Hz, H-2), 5.43 (NH, br s), 3.14 (2H, t, J=6 Hz, H-1″), 2.17 (4H, m, H-4 and H-9), 1.80 (1H, m, H-2″), 1.44 (4H, m, H-5 and H-8), 1.33 (4H, m, H-6 and H-7), 0.93 and 0.91 (3H, s, H-3″ and H-4″)

(5) C-NMR spectra [75 MHz, chloroform-d₃, δ(ppm)]: 20.11 (q, C-3″ and 4″), 28.19 (t, C-5), 28.59 (d, C-2″), 28.93 (t, C-6), 29.02 (t, C-7), 29.30 (t, C-8), 31.97 (t, C-4), 32.84 (t, C-9), 46.81 (t, C-1″), 100.89 (t, C-7′), 105.37 (d, C-2′), 108.20 (d, C-5′), 120.18 (d, C-6′), 123.60 (d, C-2), 129.32 (d, C-10 and C-11), 132.45 (s, C-1′), 144.69 (d, C-3), 146.53(s, C-4′), 147.91(s, C-3′), 166.06(s, C-1)

Compound 5

(1) Form: yellow powder

(2) Empirical formula and Molecular weight: C₁₄H₂₅NO, 223

(3) Electron impact mass spectrometry (70 eV): m/z(rel. int)=222 [M−H]+

(4) H-NMR spectra [300 MHz, chloroform-d₃, δ(ppm)]: 7.17 (1H, dd, J=14.7, 14.7 Hz, H-3), 6.08 (1H, m, H-4 and H-5), 5.76 (1H, d, J=14.7 Hz, H-2), 5.67 (NH, br s), 3.15 (2H, t, J=6.6 Hz, H-1″), 2.13 (2H, m, H-6), 1.88 (1H, m, H-2″), 1.40 (2H, m, H-7), 1.29 (4H, m, H-8 and H-9), 0.92 and 0.90 (3H, s, H-3″ and H-4″), 0.87 (3H, s, H-10)

(5) C-NMR spectra [75 MHz, chloroform-d₃, δ(ppm)]: 13.96 (q, C-10), 20.09 (q, C-3′ and C-4′), 22.43 (t, C-9), 28.44 (t, C-7), 28.59 (d, C-2′), 31.32 (t, C-8), 32.87 (t, C-6), 46.89 (t, C-1′), 121.76 (t, C-2), 128.18 (t, C-4), 141.19 (d, C-3), 143.10 (d, C-5), 166.40 (s, C-1)

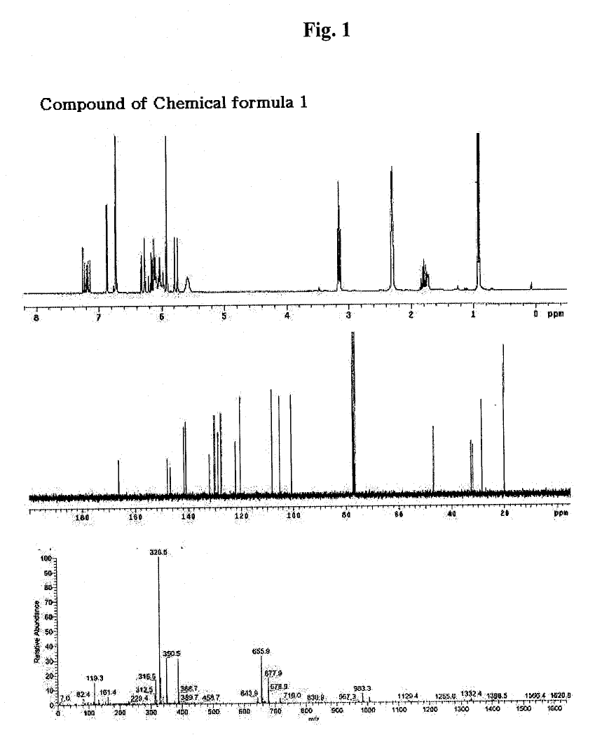

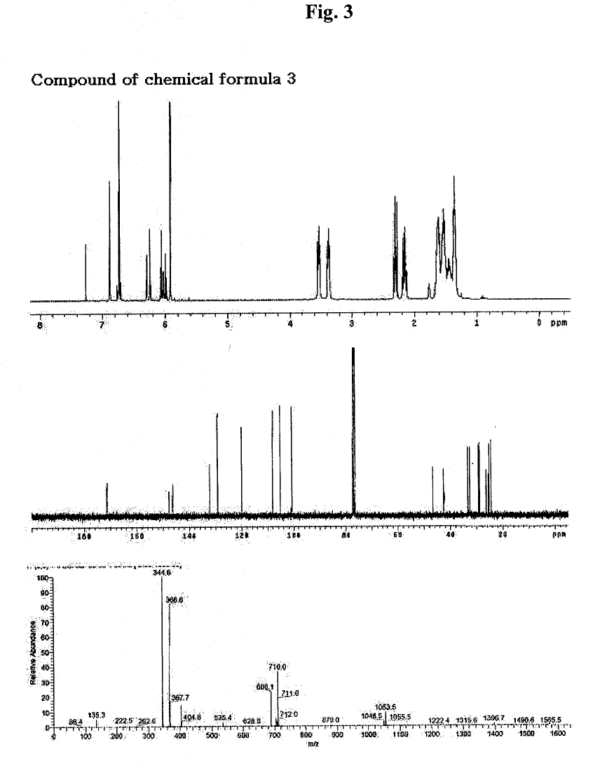

Compound 1 was completely isolated and purified as colorless crystalline powder with [M+Na]⁺ at m/z 350. It was predicted to have the empirical formula C₂₀H₂₅NO₃, as measured by high-resolution FAB-MS. In a UV spectrum, maximum absorbance was detected at 260 nm and shoulder absorbance appeared at 295˜305 nm, suggesting the presence of a conjugated dienamide in the structure of the compound. NMR was carried out to determine the structure of the compound. In ¹H-NMR spectra, one methylenedioxy proton (5.93, s) was observed and nine olefinic protons were detected at δ5.7˜7.3, the proton (br s) of —NH— at δ5.59, two methylene protons at δ2.30, one methylene proton linked to —NH— at δ3.15, one methine proton at δ1.79, and methyl protons at δ0.93 and δ0.91. From these protons, the presence of an isobutyl group could be inferred (FIGS. 1, 2 and 3). The data obtained above are very similar to those for the structure of ractamideRetrof A, which has an amide bond. By comparison and analysis with published data (Park, I. K., Lee, S. G., Shin, S. C., Park, J. D. and Ahn, Y. J. 2002. Larvicidal activity of isobutylamides identified in Piper nigrum fruits against three mosquito species: J Agric Food Chem 50, 1866-1870), Compound 1 was identified as retrofractamide A.

Compound 2 was completely isolated and purified as a colorless crystalline powder with [M-H]⁺ at m/z 354. It was predicted to have the empirical formula C₂₂H₂₉NO₃, as measured by high-resolution FAB-MS. Similar to the case of Compound 1, the UV spectrum indicated the likelihood of the presence of conjugated dienamide. ¹H-NMR spectrum data were similar to those of Compound 1, with the exception that two methylene protons were observed at δ1.46. Taken together, the data obtained above are very similar to those for the structure of pipercide, which has an amide bond. By comparison and analysis with published data (Park, I. K., Lee, S. G., Shin, S.C., Park, J. D. and Ahn, Y. J. 2002. Larvicidal activity of isobuthylamides identified in Piper nigrum fruits against three mosquito species: J Agric Food Chem 50, 1866-1870), Compound 2 was identified as pipercide.

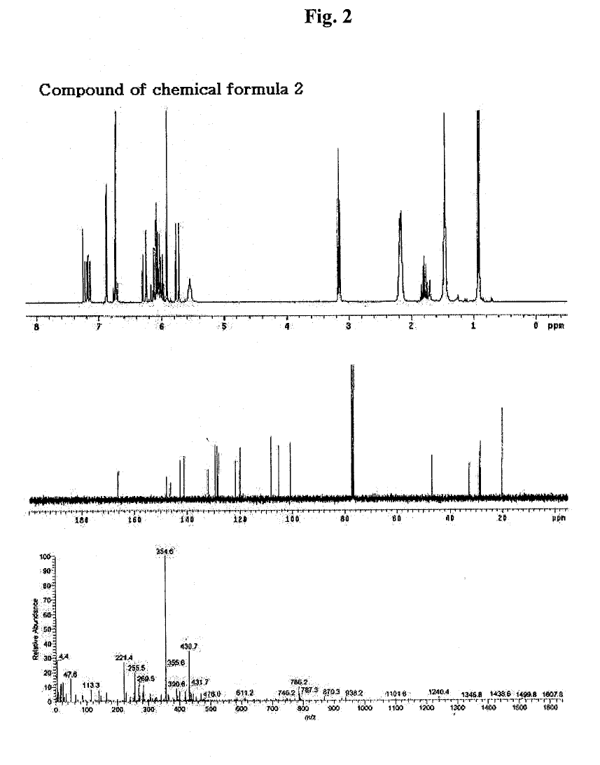

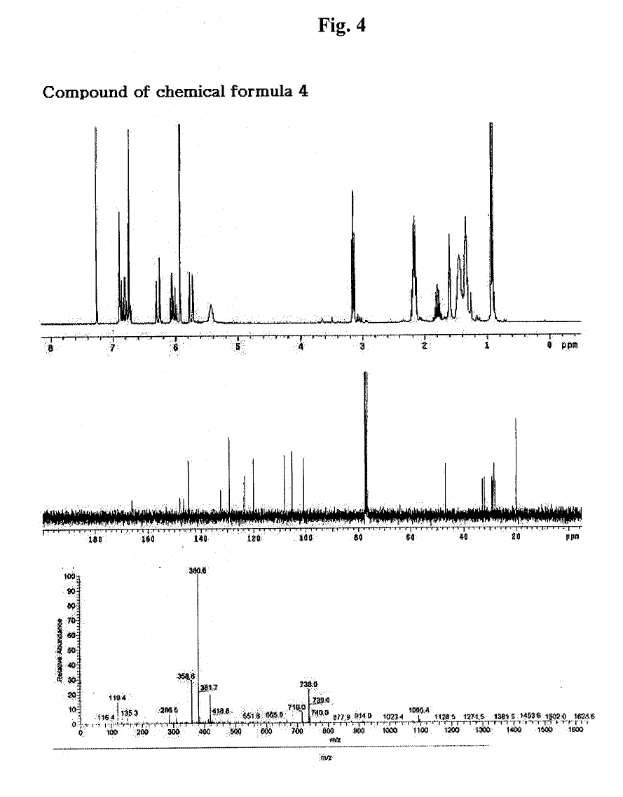

Compound 3 was completely isolated and purified as a colorless crystalline oil with [M+Na]⁺ at m/z 366. It was predicted to have the empirical formula C₂₁H₂₉NO₃, as measured by high-resolution FAB-MS. In a UV spectrum, maximum absorbance was detected at 260 nm, suggesting the presence of dienamide in the structure of the compound. NMR was carried out to determine the structure of the compound. In ¹H-NMR spectra, one methylenedioxy proton (δ5.93, s) was observed, and nine olefinic protons were detected at δ6.0˜7.0, the methylene protons (br s) bonded to —N— at δ3.54 and δ3.38, two methylene protons at δ2.30 and δ2.15, 14 methylene protons at δ1.31˜1.67 (FIGS. 2, 3 and 4). The data obtained above are very similar to those for the structure of piperrolein B, which has piperidine therein. By comparison and analysis with published data (Kiuchi, F., Nakamura, N., Tusda, Y., Kondo, K. and Yoshimura, H. 1988. Studies on crude drugs effective on visceral larva migrans. IV. isolation and identification of larvicidal principles in pepper: Chem Pharm Bull 36(7), 2452-2465), Chemical Formula 3 was identified as piperolein B.

Compound 4 was completely isolated and purified as a colorless crystalline powder with [M+Na]⁺ at m/z 380. It was predicted to have the empirical formula C₂₂H₃₁NO₃, as measured by high-resolution FAB-MS. In a UV spectrum, maximum absorbance was detected at 260 nm and shoulder absorbance appeared at 295˜305 nm, suggesting the presence of conjugated dienamide in the structure of the compound. NMR was carried out to determine the structure of the compound. In ¹H-NMR spectra, one methylenedioxy proton (5.93, s) was observed and 7 olefinic protons and 4 methylene protons were detected at δ5.7˜7.3, and at δ1.30˜1.50, respectively (FIGS. 2, 3 and 4). The data obtained above are very similar to those for the structure of piperchabamide D, which has an isobutyl group and an amide bond therein. By comparison and analysis with published data (Morikawa, T., Matsuda, H., Yamaguchi, I., Pongpiriyadacha, Y. and Yishikawa, M. 2004. New amides and gastro protective constituents from the fruit of Piper chaba: Planta Med 70, 152-159), the compound of Chemical Formula 4 was identified as piperchabamide D.

Compound 5 was completely isolated and purified as a yellow crystalline powder with [M+Na]⁺ at m/z 222. It was predicted to have the empirical formula C₁₄H₂₅NO, as measured by high-resolution FAB-MS. In a UV spectrum, the maximum absorbance was detected at 260 nm and shoulder absorbance appeared at 295˜305 nm, suggesting the presence of conjugated dienamide in the structure of the compound. NMR was carried out to determine the structure of the compound. In ¹H-NMR spectra, 4 olefinic protons were observed at δ5.7˜7.3, the proton of —NH— (br, s) at δ5.67, one methylene proton at each of δ3.15 and δ2.13, three methylene protons at δ1.20˜1.45, a methine proton at δ1.88, and three methyl protons at δ0.87, δ0.90 and δ0.92. Based on the data obtained above, Compound 5 was predicted to be pellitorine having an isobutyl group, but not a methylenedioxybenzyl group. By comparison and analysis with published data (Park, I. K., Lee, S. G., Shin, S. C., Park, J. D. and Ahn, Y. J. 2002. Larvicidal activity of isobutylamides identified in Piper nigrum fruits against three mosquito species: J Agric Food Chem 50, 1866-1870), the compound of Chemical Formula 5 was identified as pellitorin.

### EXAMPLE 3

### Preparation of ACAT Source

The livers were removed from Male Sprague-Dawley rats (250-300 g), washed with microsome buffer A (0.25 M sucrose, 1 mM EDTA, 0.01 M Tris-HCl, pH 7.4), finely fractioned with scissors, and homogenized using a Teflon-glass homogenizer. The homogenate was centrifuged at 14,000× g for 15 min to give a supernatant which was the further centrifuged at 100,000× g for 1 hour. The pellet thus obtained was dissolved in microsome buffer B (0.25 M sucrose, 0.01 M Tris-HCl, pH 7.4) before centrifugation at 100,000× g for 1 hour to separate ACAT-containing microsomes. In order to obtain uniform protein concentrations of the enzyme source, the pellet was dissolved in a suitable amount of microsome buffer B, and assayed for protein concentration by the Lowry method with BSA (bovine serum albumin) as a standard. Thereafter, the enzyme source thus obtained was diluted with microsome buffer B to a protein concentration of 10 mg/ml, aliquoted in 1 ml vials, and stored at −70° C. until use.

### EXAMPLE 4

### Assay for ACAT Activity

ACAT activity was assayed with [1-¹⁴C]oleoyl-CoA as a substrate using a modification of Kim's method [Kim Y. K, H. W. Lee, K. H Son, B. M Kwon, T. S Jeong, D. H. Lee, J. H. Shin, Y. W. Seo, S. U. Kim, and S. H. Bok 1996. GERI-BP002-A, Novel inhibitors of acyl-CoA:cholesterol acyltransferase produced by Aspergillus fumigatus F93: J. Antibiotics 49:31-36]. A reaction solution was prepared by mixing 10.0 μl of the sample, 4.0 μl of a rat liver microsomal enzyme of rat, 20.0 μl of an assay buffer [0.5 M KH₂PO₄, 10 mM DTT, pH 7.4], 15.0 μl of 40 mg/me BSA (essentially fatty acid free), 2.0 μl of 20 mg/me cholesterol, and 41.0 μl of distilled water, and subjected to pre-reaction at 37° C. for 20 min. To this enzymatic solution was added 8.0 μl of [1-¹⁴C]oleoyl-CoA (0.05 μCi, final conc. 10 μM), and enzyme reaction was carried out at 37° C. for 25 min and terminated with 1 ml of isopropanol-heptane (4:1 v/v). Following the addition of 0.6 ml of heptane and 0.4 ml of five fold-diluted assay buffer, centrifugation was performed to separate the organic solvent. 100 μl of the supernatant was mixed well with 3 ml of Lipoluma scintillation cocktail and radioactivity was measured using a liquid scintillation counter.

Activity of inhibiting ACAT was quantified as radioactivity detected in the product of the reaction between the radio-labeled substrate and the enzyme in the presence of the assay samples, and percentage activity inhibition was calculated according to Equation 1 as follows.

% Activity Inhibition=100×[1-CPM(T)-CPM(C2)/CPM(C1)-CPM(B)] [Equation 1]

CPM (T): CPM detected upon co-existence of sample and enzyme

CPM (C1): CPM upon existence of enzyme alone in the absence of sample

CPM (C2): CPM detected upon existence of sample alone in the absence of enzyme

CPM (B): CPM detected in the absence of both enzyme and sample.

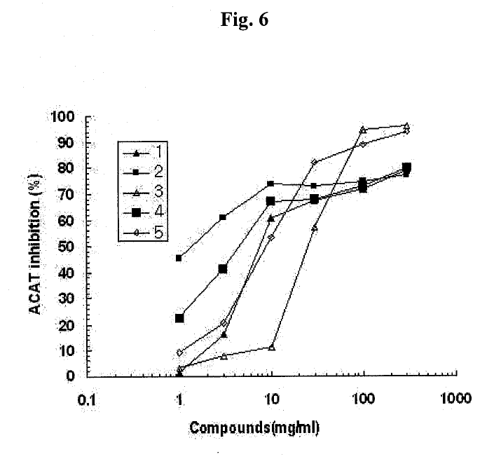

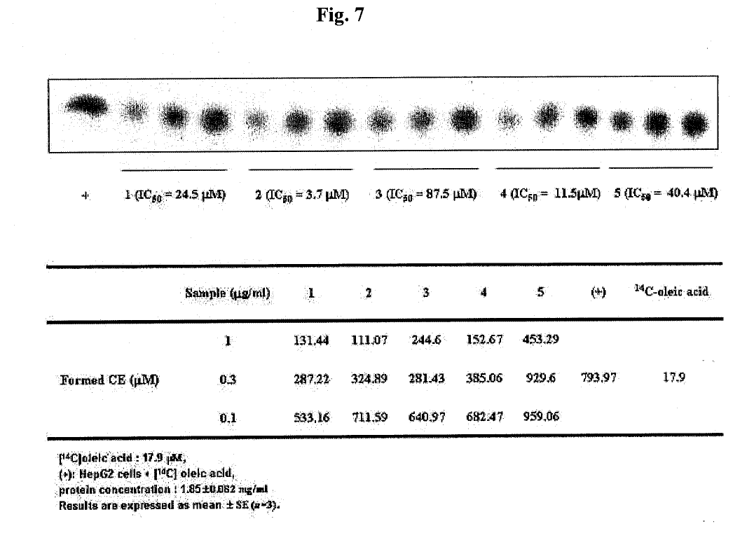

A blank sample was reacted at 0° C. While obovatol, serving as a positive control, was measured to have an IC₅₀ value of 44 μM against ACAT, the amide compounds of Chemical Formulas 1 to 5 have IC₅₀ values of 24.5, 3.7, 87.5, 11.5, and 40.4 μM, respectively, showing inhibition activity in a dose-dependent manner (FIGS. 6 and 7).

Having the function of interrupting the absorption of cholesterol in the small intestine, thus decreasing serum levels of cholesterol, hindering the synthesis of VLDL in the liver to thus decrease serum LDL cholesterol, and inhibiting the acylation of cholesterol, essential for the advance of arteriosclerosis at affected lesions, the ACAT inhibitors can be used as effective medications for the prevention and treatment of high cholesterol level-associated vascular diseases such as hyperlipidemia, arteriosclerosis, etc.

### EXAMPLE 5

### Preparation of Tablet

Pipercide—1 g

Lactose—7 g

Crystalline cellulose—1.5 g

Magnesium stearate—0.5 g

Total—10 g

The above ingredients were mixed well and formulated into tablets using a direct tableting method. Each tablet weighed 100 mg and contained 10 mg of the active ingredient.

### EXAMPLE 6

### Preparation of Powder

Pipercide—1 g

Corn starch—5 g

Carboxyl cellulose—4 g

Total—10 g

These ingredients were mixed well to give powder which was then encapsulated in a soft capsule in an amount of 100 mg per capsule.

### INDUSTRIAL APPLICABILITY

As described hitherto, retrofractamide A, pipercide, piperrolein B, piperchabamide D, pellitorin, and pharmaceutically acceptable salts thereof effectively inhibit ACAT and thus can be used, alone or in combination, for the prevention and treatment of vascular diseases, such as hyperlipidemia, arteriosclerosis, etc.

In addition, an extract from Piper nigrum L. or fractions separated from the extract exhibit the same inhibitory activity as described above because the extract or the fractions, although unpurified, contain all of the active ingredients. Therefore, the extract can be effective used in pharmaceutical compositions having activity of inhibiting ACAT as well as in heath foods useful for the prevention of vascular diseases.

1. A composition having activity of inhibiting acyl-CoA:cholesterol acyltransferase, comprising an extract from Piper nigrum L. or a non-polar organic solvent fraction 5 thereof.

2. The composition as defined in claim 1, wherein the extract from piper nigrum L. is prepared using water, an organic solvent or mixtures thereof.

3. The composition as defined in claim 2, wherein the organic solvent is selected from a group consisting of methanol, ethanol, isopropanol, butanol, ethylene, acetone, hexane, ether, chloroform, ethylacetate, butyl acetate, dichloromethane, N,N-dimethylformamide (DMF), dimethylsulfoxide (DMSO), 1,3-butyleneglycol, propyleneglycol, and combinations thereof.

4. The composition as defined in claim 3, wherein the organic solvent is selected from a group consisting of methanol, ethanol, isopropanol, butanol, and combinations thereof.

5. The composition as defined in claim 1, wherein the non-polar solvent fraction is prepared by dissolving the extract in a non-polar solvent selected from a group consisting of n-hexane, ether, dichloromethane, chloroform, ethyl acetate and combinations thereof.

6. A method for preparing an extract having activity of inhibiting acyl-CoA:cholesterol acyltransferase from Piper nigrum L., comprising extracting active materials from Piper nigrum L. into water, an organic solvent or a mixture thereof through cold precipitation or heat extraction.

7. The method as defined in claim 6, further comprising fractioning the extract in a solvent selected from a group consisting of hexane, ether, dichloromethane, chloroform, ethylacetate and combinations thereof.

8. A pharmaceutical composition for the prevention and treatment of vascular diseases and Alzheimer's disease, comprising the composition of one of claims 1 to 5.

9. A health aid composition, comprising the composition of one of claims 1 to 5.

10. A composition having activity of inhibiting acyl-CoA:cholesterol acyltransferase, comprising at least one of the compounds represented by the following chemical formulas 1 to 5, or at least one of pharmaceutically acceptable salts thereof.

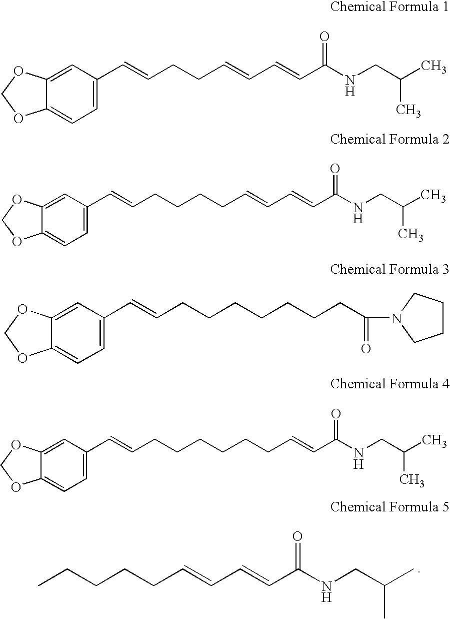

11. The composition as defined in claim 10, wherein the composition is for use in prevention and treatment of vascular disease.

12. The composition as defined in claim 11, wherein the vascular disease is cardiovascular disease or peripheral vascular disease.

13. The composition as defined in claim 12, wherein the cardiovascular disease or the peripheral vascular disease is selected from a group consisting of hypercholesterolemia, hyperlipidemia, atherosclerosis, arteriosclerosis, coronary arteriosclerosis, aortic aneurysms, and combinations thereof.

14. The composition as defined in claim 10, wherein the composition is for use in the prevention and treatment of Alzheimer's disease.

15. A method for separating one of the compounds represented by Chemical Formulas 1 to 5 in claim 10, comprising:
a) preparing an extract from Piper nigrum L. in water, an organic solvent or mixtures thereof;
b) fractioning the extract in water or a non-polar organic solvent; and
c) isolating and purifying the compounds from the fraction.

16. The method as defined in claim 15, wherein the organic solvent used in step a) is selected from a group consisting of methanol, ethanol, isopropanol, butanol, ethylene, acetone, hexane, ether, chloroform, ethyl acetate, butyl acetate, dichloromethane, N,N-dimethylformamide (DMF), dimethylsulfoxide (DMSO), 1,3-butyleneglycol, propyleneglycol and combinations thereof.

17. The method as defined in claim 16, wherein the organic solvent is selected from a group consisting of methanol, ethanol, isopropanol, butanol, or a mixture thereof.

18. The method as defined in claim 15, wherein the non-polar organic solvent used in step b) is selected from a group consisting of hexane, ether, dichloromethane, chloroform, ethylacetate, or a mixture thereof.

19. The method as defined in claim 15, wherein the non-polar organic solvent is chloroform or ethylacetate.

20. The method as defined in claim 15, wherein the step c) is performed through chromatography.
